(19) United States
(12) Patent Application Publication
(10) Pub. No.: US 2009/0105696 A1
(43) Pub. Date: Apr. 23, 2009

(54) ND:YAG LASER FOR REMOVING FATTY TISSUE

(75) Inventors: Hee Chul Lee, Gyeonggi-do (KR); Hae Lyung Hwang, Gyeonggi-do (KR)

Correspondence Address: PEPPER HAMILTON LLP, ONE MELLON CENTER, 50TH FLOOR, 500 GRANT STREET, PITTSBURGH, PA, 15219, US

(73) Assignee: LUTRONIC CORPORATION, Gyeonggi-do (KR)

(21) Appl. No.: 12/280,460

(22) PCT Filed: Feb. 15, 2007
(86) PCT No.: PCT/KR07/00800

§ 371 Date: Nov. 20, 2008

(30) Foreign Application Priority Data
Feb. 22, 2006 (KR) 10-2006-0017383

(51) Int. Cl. A61B 18/20

(52) U.S. Cl. 606/3

(57) ABSTRACT

A Nd:YAG laser at a wavelength of 1444 nm for removing fatty tissue is disclosed, which can directly irradiate laser light onto subcutaneous fat and efficiently remove fatty tissue with an adverse reaction on surrounding tissue minimized by using laser light at an oscillating wavelength of 1444 nm which has both a high fat absorptivity and a high water absorptivity among wavelengths that can be oscillated by the Nd:YAG laser. The Nd:YAG laser includes a flash lamp, a Nd:YAG rod, a high reflection mirror, an output coupler, a convergent lens for converging the laser light beam oscillating through the output coupler, an optical fiber for guiding the converged laser beam, and a guide needle for guiding the laser beam from the guide fiber to subcutaneous fat. Both end surfaces of the Nd:YAG rod, an inner surface of the high reflection mirror, and inner and outer surfaces of the output coupler are coated so as to oscillate only the laser beam at the wavelength of 1444 nm through the output coupler.

### TECHNICAL FIELD

The present invention relates to a Nd (neodymium):YAG (yttrimim aluminum garnet) laser for removing fatty tissue, and more particularly to a Nd:YAG laser at a wavelength of 1444 nm for removing fatty tissue, which can directly irradiate laser light onto subcutaneous fat without irradiating the laser light from an outside of a skin, and can efficiently remove fatty tissue with an adverse reaction on surrounding tissue minimized by using laser light at an oscillating wavelength of 1444 nm which has both a high fat absorptivity and a high water absorptivity among wavelengths that can be oscillated by the Nd:YAG laser.

### BACKGROUND ART

FIG. 1 is an analytic view illustrating water and fat absorptivities of laser light. As illustrated in FIG. 1, the wavelength of light that fatty tissue absorbs at maximum is in the neighborhood of 920 nm, 1200 nm, 1750 nm, and 1410 nm. However, in consideration of the water absorptivity of the laser light, the laser light at a wavelength of 1200 nm is most ideal.

The current Nd:YAG laser does not oscillate at a wavelength of 1200 nm, but its main oscillation wavelength is in the range of 946 nm, 1064 nm, 1320 nm, 1340 nm, and 1444 nm.

Accordingly, a conventional fat removing method removes fatty tissue using laser light at a wavelength of 1064 nm which is the highest output of the Nd:YAG laser, and has a low fat absorptivity and a high water absorptivity.

However, the conventional fat removing method using the Nd:YAG laser has the following problems.

According to the conventional fat removing method using the Nd:YAG laser oscillating at a wavelength of 1064 nm, the laser light at the wavelength of 1064 nm has a low fat absorptivity as shown in FIG. 1, and thus the laser light is spread to tissue adjacent to the fatty tissue when the fatty tissue is removed.

If the laser beam at a wavelength of 1064 nm is spread to the tissue adjacent to the fat tissue, it damages the tissue except for the fatty tissue because the laser beam at a wavelength of 1064 also has a low water absorptivity.

That is, in the case of using the laser beam at the wavelength of 1064 nm, the fat removing itself does not go well, and the surrounding tissue is seriously damaged.

Even if the laser light in the neighborhood of a wavelength of 1200 nm, which has a high fat absorptivity but has a low water absorptivity, is used, it has a low water absorptivity as shown in FIG. 3, and thus it may greatly damage the adjacent tissue when a user irradiates the laser beam into the adjacent tissue by mistake.

### DISCLOSURE OF INVENTION

### Technical Problem

Therefore, the present invention has been made in view of the above-mentioned problems, and it is an object of the present invention to provide a Nd:YAG laser at a wavelength of 1444 nm for removing fatty tissue, which can directly irradiate laser light onto subcutaneous fat by directly inserting an optical fiber and a guide needle into the subcutaneous fat and outputting laser light having a wavelength that maximizes a fat absorptivity only in consideration of the fat absorptivity, without considering a loss of laser energy due to water absorptivity of the laser light.

It is another object of the present invention to provide a Nd:YAG laser at a wave length of 1444 nm for removing fatty tissue, which can efficiently remove fatty tissue with damage of surrounding tissue minimized by oscillating laser light at a wavelength of 1444 nm which has both a high fat absorptivity and a high water absorptivity among laser lights that can be oscillated by the Nd:YAG laser.

### Technical Solution

In order to achieve the above object, in one aspect of the present invention, there is provided a Nd:YAG (Neodymium:Yttrimium Aluminum Garnet) laser at a wavelength of 1444 nm that directly irradiates laser light onto fatty tissue to remove the fatty tissue and includes a flash lamp for being supplied with a power from a power supply unit and emitting light, a Nd:YAG rod for amplifying and oscillating the excited laser light inputted from the flash lamp, a high reflection mirror and an output coupler, positioned on both sides of the Nd:YAG rod, for reflecting the light outputted from the Nd:YAG rod, according to the present invention, which comprises a convergent lens for converging the laser light beam oscillating through the output coupler; an optical fiber for guiding the laser beam converged by the convergent lens; and a guide needle, made of metallic material and connected to an end of the optical fiber, for guiding the laser beam from the guide fiber to subcutaneous fat; wherein both end surfaces of the Nd:YAG rod, an inner surface of the high reflection mirror, and inner and outer surfaces of the output coupler are coated so as to oscillate only the laser beam at the wavelength of 1444 nm through the output coupler.

### Advantageous Effects

The Nd:YAG laser at a wavelength of 1444 nm for removing fatty tissue as constructed above can directly irradiate laser light onto subcutaneous fat and efficiently remove fatty tissue with an adverse reaction on surrounding tissue minimized by using laser light at an oscillating wavelength of 1444 nm which has both a high fat absorptivity and a high water absorptivity among wavelengths that the Nd:YAG laser can oscillate.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is an analytic view illustrating water and fat absorptivities of laser light;

FIG. 2 is a conceptual view explaining a case in which laser light at a wavelength of 1064 nm is used according to the conventional technique;

FIG. 3 is a conceptual view explaining a case in which laser light at a wavelength in the neighborhood of 1200 nm is used;

FIG. 4 is a view illustrating the construction of a Nd:YAG laser at a wavelength of 1444 nm that directly irradiates laser light onto fatty tissue to remove the fatty tissue according to an embodiment of the present invention; and

FIG. 5 is a conceptual view explaining a case in which laser light at a wavelength of 1444 nm is used according to the present invention.

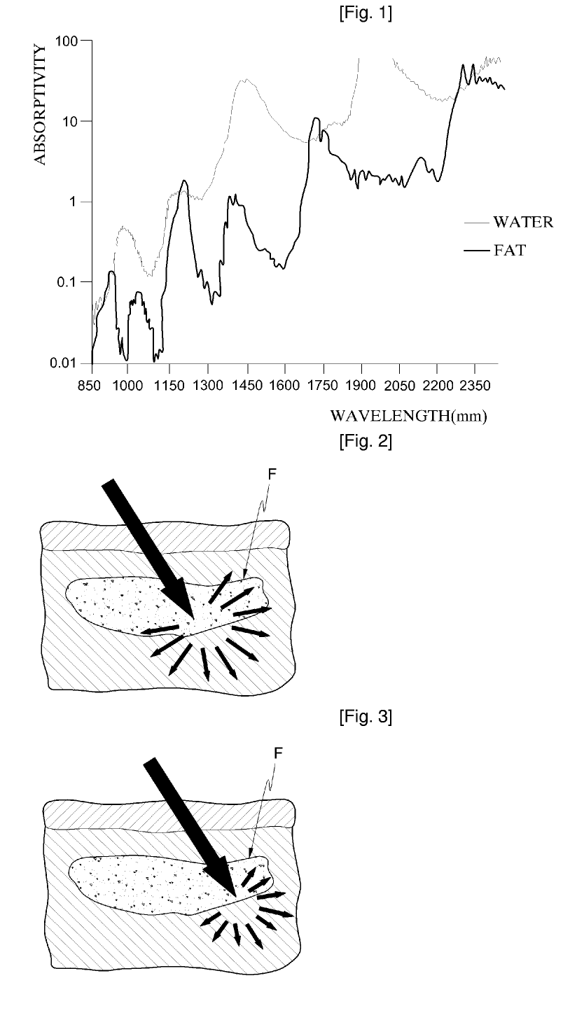

### BEST MODE FOR CARRYING OUT THE INVENTION

Hereinafter, preferred embodiments of the present invention will be described with reference to the accompanying drawings. The matters defined in the description, such as the detailed construction and elements, are nothing but specific details provided to assist those of ordinary skill in the art in a comprehensive understanding of the invention, and thus the present invention is not limited thereto.

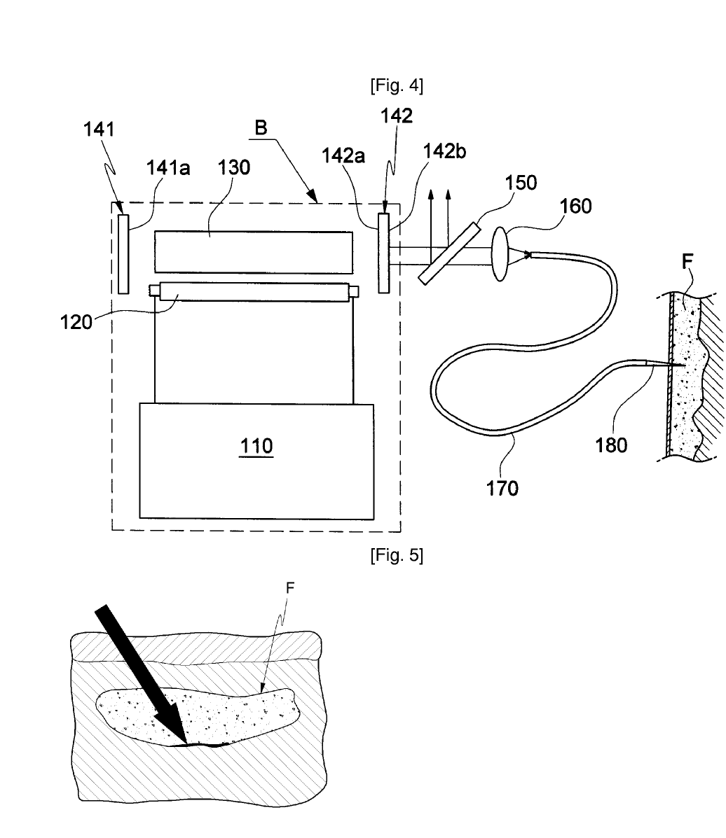

FIG. 4 is a view illustrating the construction of a Nd:YAG laser at a wavelength of 1444 nm that directly irradiates laser light onto fatty tissue to remove the fatty tissue according to an embodiment of the present invention.

As shown in FIG. 4, the Nd:YAG laser at a wavelength of 1444 nm that directly irradiates laser light onto fatty tissue to remove the fatty tissue according to an embodiment of the present invention includes a Nd:YAG laser main body B, a filter 150, an optical fiber 170, and a guide needle 180.

The Nd:YAG laser main body B includes a flash lamp for being supplied with a power from a power supply unit 110 and emitting light, a Nd:YAG rod 130 for amplifying and oscillating excited laser light inputted from the flash lamp 120, a high reflection mirror 141 and an output coupler 142, positioned on both sides of the Nd:YAG rod 130, for reflecting the light outputted from the Nd:YAG rod 130.

A convergence lens 160 converges the laser light beam oscillating through the output coupler 142, and comprises a convex lens.

The optical fiber 170 receives the beam converged by the convergent lens 160, and guides the incident beam inside subcutaneous fat F.

The guide needle 180 is coupled to an end part of the optical fiber 170, and an end of the guide needle 180 is inserted into the subcutaneous fat F to guide the beam through the optical fiber 170 to the subcutaneous fat F.

It is preferable that the guide needle 180 is made of metallic material, and is constructed to guide the beam having been guided by the optical fiber 170 to the sub-cutaneous fat more conveniently and simply.

Since the laser light is directly irradiated onto the subcutaneous fat through the optical fiber 170 and the guide needle 180, a loss of laser light due to the absorption of the laser light into water, which may occur in the case of irradiating the laser beam from the outside of the skin, is prevented, and thus the laser light having a wavelength of 1444 nm that has a high fat absorptivity can be used to remove the subcutaneous fat.

In order to make the laser beam that is outputted through the output coupler 142 oscillate at a wavelength of 1444 nm, both end surfaces of the Nd:YAG rod 130, an inner surface 141a of the high reflection mirror 1441, and inner and outer surfaces 142a and 142b of the output coupler 142 are coated.

Specifically, in order to oscillate only the laser beam at a wavelength of 1444 nm, the both surfaces of the Nd:YAG rod 130 are antireflection-coated so as to effect an antireflection of the light at a wavelength of 1000-1500 nm. The inner surface 141a of the high reflection mirror 141 is coated to fully reflect the light at a wavelength of 1444 nm and to transmit over 95% of the light at wavelengths in the range of 1320-1340 nm and 1064 nm. The inner surface 142a of the output coupler 142 is coated to have a reflection rate of 80% at a wavelength in the range of 1300-1500 nm and to have a transmission rate of over 95% at a wavelength of 1064 nm, and the outer surface of the output coupler 142 is antireflection-coated at a wavelength band of 1000-1500 nm.

As described above, the reason why to oscillate and use only the laser beam with a wavelength of 1444 nm in order to remove the fatty tissue is that the laser beam at the wavelength of 1444 nm has the highest fat absorptivity among the laser beams that the Nd:YAG laser can oscillate.

In the case of using the laser beam at a wavelength of 1444 nm, the laser beam may not be fully absorbed into the fatty tissue, but may propagate through the surrounding tissue. However, since the laser beam at the wavelength of 1444 nm has a high water absorptivity, the surrounding tissue is a little damaged in comparison to the case that the laser beam at other wavelengths is used.

The filter 150, which is installed in front of the convergent lens 160, reflects the laser beam at a wavelength in the range of 1060˜1350 nm and transmits only the laser beam at a wavelength of 1444 nm in order to prevent the laser light at a wavelength of 1064 nm among the laser lights outputted from the output coupler 142 from being incident to the optical fiber 170.

The output of the laser used in the present invention has the repetition rate of 1-20 Hz, energy per pulse of 100˜1000 mJ, power of 0.5-20W, and pulse width that varies in the range of 100 μs˜60 ms.

The operation of the Nd:YAG laser at a wavelength of 1444 nm that directly irradiates laser light onto fatty tissue to remove the fatty tissue as constructed above according to an embodiment of the present invention will now be described in detail.

FIG. 5 is a conceptual view explaining a case in which laser light at a wavelength of 1444 nm is used according to the present invention.

As described above, the both surfaces of the Nd:YAG rod 130 are antireflection-coated so as to effect an antireflection of the light at a wavelength of 1000˜1500 nm. The inner surface 141a of the high reflection mirror 141 is coated to fully reflect the light at a wavelength of 1444 nm and to transmit over 95% of the light at wavelengths in the range of 1320˜1340 nm and 1064 nm. The inner surface 142a of the output coupler 142 is coated to have a reflection rate of 80% at a wavelength in the range of 1300˜1500 nm and to have a transmission rate of over 95% at a wavelength of 1064 nm, and the outer surface of the output coupler 142 is antireflection-coated at a wavelength band of 1000˜1500 nm.

If a power is supplied to the power supply unit, the output coupler 142 outputs only the laser beam at a wavelength of 1444 nm.

In this case, the laser beam at a wavelength of 1064 nm, which may be minutely generated when the output of the output coupler 142 becomes high, is filtered by the filter 150, and thus cannot be incident to the optical fiber 170.

The laser beam at a wavelength of 1444 nm, having passed through the filter 150, is converged by the convergent lens 160, is guided by the optical fiber 170 and the guide needle 180, and then is directly irradiated onto the subcutaneous fat F to split the fatty tissue.

As shown in FIG. 5, the laser light at a wavelength of 1444 nm used in the present invention has both a high fat absorptivity and a high water absorptivity, and thus even in the case where the user irradiates the laser beam onto the tissue neighboring the sub-cutaneous fat by mistake, water in the human body absorbs the laser to prevent the expansion of heat. Accordingly, even if the user irradiates the laser beam onto the tissue neighboring the fatty tissue by mistake, the damage of the neighboring tissue (indicated by a thin black line in FIG. 5) can be minimized.

### INDUSTRIAL APPLICABILITY

As can be seen from the foregoing, the Nd:YAG laser at a wavelength of 1444 nm that directly irradiates laser light onto fatty tissue to remove the fatty tissue according to an embodiment of the present invention has the following effects.

First, since only the laser beam at a wavelength of 1444 nm, which has the highest fat absorptivity among laser beams at other wavelengths that the Nd:YAG laser can oscillate, is oscillated and used to remove the fatty tissue, the Nd:YAG laser has a superior effect of removing the fatty tissue.

Second, even in the case that the laser beam at a wavelength of 1444 nm is not fully absorbed into the fatty tissue, but propagates through the surrounding tissue, the damage of the surrounding tissue is minimized since the laser beam at the wavelength of 1444 nm also has a high water absorptivity.

While this invention has been described in connection with what is presently considered to be the most practical and preferred embodiment, it is to be understood that the invention is not limited to the disclosed embodiment and the drawings, but, on the contrary, it is intended to cover various modifications and variations within the spirit and scope of the appended claims.

1. A Nd: YAG (Neodymium:Yttrimium Aluminum Garnet) laser at a wavelength of 1444 nm that directly irradiates laser light onto fatty tissue to remove the fatty tissue and includes a flash lamp for being supplied with a power from a power supply unit and emitting light, a Nd: YAG rod for amplifying and oscillating the excited laser light inputted from the flash lamp, a high reflection mirror and an output coupler, positioned on both sides of the Nd:YAG rod, for reflecting the light outputted from the Nd: YAG rod, the Nd: YAG laser comprising:
a convergent lens for converging the laser light beam oscillating through the output coupler;
an optical fiber for guiding the laser beam converged by the convergent lens; and
a guide needle, made of metallic material and connected to an end of the optical fiber, for guiding the laser beam from the guide fiber to subcutaneous fat;
wherein both end surfaces of the Nd: YAG rod, an inner surface of the high reflection mirror, and inner and outer surfaces of the output coupler are coated so as to oscillate only the laser beam at the wavelength of 1444 nm through the output coupler.

2. The Nd: YAG laser of claim 1, wherein in order to oscillate only the laser beam at a wavelength of 1444 nm, both surfaces of a Nd: YAG rod are antireflection-coated so as to effect an antireflection of the light at a wavelength of 1000˜1500 nm;
an inner surface of the high reflection mirror is coated to fully reflect the light at a wavelength of 1444 nm and to transmit over 95% of the light at wavelengths in the range of 1320˜1340 nm and 1064 nm;
an inner surface of the output coupler is coated to have a reflection rate of 80% at a wavelength in the range of 1300˜1500 nm and to have a transmission rate of over 95% at a wavelength of 1064 nm; and
an outer surface of the output coupler is antireflection-coated at a wavelength band of 1000˜1500 nm.

3. The Nd: YAG laser of claim 2, further comprising a filter, installed in front of the convergent lens, for reflecting the laser beam at a wavelength in the range of 1060˜1350 nm and transmitting only the laser beam at a wavelength of 1444 nm in order to prevent the laser light at a wavelength of 1064 nm among the laser lights outputted from the output coupler from being incident to the optical fiber.

4. The Nd: YAG laser of claim 1, wherein the oscillating laser beam output has a repetition rate of 1-20 Hz, energy per pulse of 100˜1000 mJ, power of 0.5-20W, and pulse width that varies in the range of 100 μs˜60 ms.

5. The Nd: YAG laser of claim 2, wherein the oscillating laser beam output has a repetition rate of 1-20 Hz, energy per pulse of 100˜1000 mJ, power of 0.5-20W, and pulse width that varies in the range of 100 μs˜60 ms.

6. The Nd: YAG laser of claim 3, wherein the oscillating laser beam output has a repetition rate of 1-20 Hz, energy per pulse of 100˜1000 mJ, power of 0.5-20W, and pulse width that varies in the range of 100 μs˜60 ms.
